(19) United States
(12) Patent Application Publication
(10) Pub. No.: US 2016/0287394 A1
(43) Pub. Date: Oct. 6, 2016

(54) ORTHOPAEDIC IMPLANT HAVING A BIOCOMPATIBLE INDICIA APPLIED TO A POROUS SURFACE THEREOF

(71) Applicant: Larry G. McCleary, Warsaw, IN (US)

(72) Inventor: Larry G. McCleary, Warsaw, IN (US)

(21) Appl. No.: 14/674,670

(22) Filed: Mar. 31, 2015

(51) Int. Cl. A61F 2/34; A61F 2/40; A61F 2/42; A61F 2/38

(52) U.S. Cl. CPC A61F 2/34; A61F 2/38; A61F 2/40; A61F 2/4202; A61F 2210/0004; A61F 2250/0086; A61F 2250/0089

(57) ABSTRACT

An orthopaedic hip implant includes an acetabular cup having a rounded outer surface configured to be implanted into a surgically-prepared surface of a patient's acetabulum. A porous metallic coating is disposed on the rounded outer surface of the acetabular cup, with a hydroxyapatite coating being disposed on the porous metallic coating in a pattern which defines a readable indicia.

### TECHNICAL FIELD

The present disclosure relates generally to orthopaedic implants, and more particularly to orthopaedic implants having a biocompatible indicia applied to a porous surface thereof.

### BACKGROUND

During the lifetime of a patient, it may be necessary to perform a joint replacement procedure on the patient as a result of, for example, disease or trauma. In such procedure, a number of metallic implants are used to replace the compromised natural bone structure of the joint being surgically replaced. Such metallic implants are often coated with a porous metallic coating to enhance bone attachment and facilitate bony ingrowth.

Metallic implants are generally marked with indicia (both human-readable and machine-readable) to identify the implant (manufacturer, lot code, implant size, etcetera) for, amongst other things, regulatory requirements.

### SUMMARY

According to one aspect, an orthopaedic hip implant includes an acetabular cup having a rounded outer surface configured to be implanted into a surgically-prepared surface of a patient's acetabulum. A porous metallic coating is disposed on the rounded outer surface of the acetabular cup, with a hydroxyapatite coating being disposed on the porous metallic coating in a pattern which defines a readable indicia.

In an embodiment, the hydroxyapatite coating is disposed on the porous metallic coating in a pattern which defines a human-readable indicia.

In a more specific embodiment, the hydroxyapatite coating is disposed on the porous metallic coating in a pattern which defines a human-readable indicia indicative of the size of the acetabular cup.

The hydroxyapatite coating may be disposed on the porous metallic coating in a pattern which defines a machine-readable indicia.

In a more specific embodiment, the machine-readable indicia may include a barcode.

In a specific embodiment, the hydroxyapatite coating includes a plasma-sprayed hydroxyapatite coating.

According to another aspect, an orthopaedic implant includes a metallic joint prosthesis having an outer surface configured to be implanted into a surgically-prepared surface of a bone of a patient. A porous metallic coating is disposed on the outer surface of the joint prosthesis, with a hydroxyapatite coating being disposed on the porous metallic coating in a pattern which defines a readable indicia.

In an embodiment, the hydroxyapatite coating is disposed on the porous metallic coating in a pattern which defines a human-readable indicia indicative of the size of the metallic joint prosthesis.

The hydroxyapatite coating may be disposed on the porous metallic coating in a pattern which defines a machine-readable indicia.

In an embodiment, the machine-readable indicia may include a barcode.

The hydroxyapatite coating may include a plasma-sprayed hydroxyapatite coating.

The metallic joint prosthesis may be selected from the group consisting of a hip implant, a knee implant, a shoulder implant, and an ankle implant.

According to another aspect, an orthopaedic hip implant includes an acetabular cup having a rounded outer surface configured to be implanted into a surgically-prepared surface of a patient's acetabulum. A porous metallic coating is disposed on the rounded outer surface of the acetabular cup, with a resorbable coating being disposed on the porous metallic coating in a pattern which defines a readable indicia.

In an embodiment, the resorbable coating is disposed on the porous metallic coating in a pattern which defines a human-readable indicia.

In a more specific embodiment, the resorbable coating is disposed on the porous metallic coating in a pattern which defines a human-readable indicia indicative of the size of the acetabular cup.

The resorbable coating may be disposed on the porous metallic coating in a pattern which defines a machine-readable indicia.

In a more specific embodiment, the machine-readable indicia may include a barcode.

In a specific embodiment, the hydroxyapatite coating includes a plasma-sprayed hydroxyapatite coating.

### BRIEF DESCRIPTION OF THE DRAWINGS

The detailed description particularly refers to the following figures, in which:

FIG. 1 is an exploded perspective view of an implantable hip prosthesis;

FIG. 2 is a perspective view of the acetabular cup of the hip prosthesis of FIG. 1 showing a human-readable indicia disposed on the cup's porous metallic coating;

FIG. 3 is an enlarged fragmentary view of FIG. 2 showing the human-readable indicia in more detail; and

FIG. 4 is a view similar to FIG. 2, but showing a machine-readable indicia disposed on the cup's porous metallic coating.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention.

Terms representing anatomical references, such as anterior, posterior, medial, lateral, superior, inferior, etcetera, may be used throughout this disclosure in reference to both the orthopaedic implants described herein and a patient's natural anatomy. Such terms have well-understood meanings in both the study of anatomy and the field of orthopaedics. Use of such anatomical reference terms in the specification and claims is intended to be consistent with their well-understood meanings unless noted otherwise.

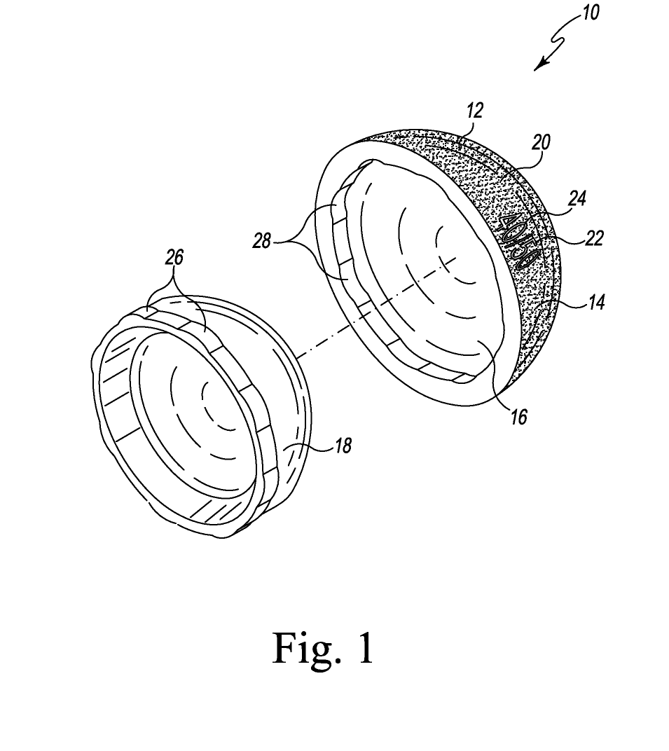

Referring to FIG. 1, an orthopaedic implant 10 is shown. In the illustrative embodiment described herein, the orthopaedic implant is embodied as an acetabular cup 12 of a hip prosthesis. The acetabular cup 12 is configured to be implanted into a surgically-prepared (e.g., reamed) surface of the acetabulum of a patient. The acetabular cup 12 includes an outer surface 14 having a generally rounded shape that is hemispherical or at least partially spherical.

Opposite its rounded outer surface 14, the acetabular cup 12 includes an inner surface 16 that is concave in shape. The inner surface 16 defines a cavity into which a polymeric bearing insert 18 is secured. The bearing insert 18 provides an artificial articular surface for a natural or artificial head of the femur of the patient. To this end, the bearing insert 18 may be formed from a polymeric material such as polyethylene or, more specifically, ultra-high molecular weight polypropylene (UHMWPE). The acetabular cup 12 and the bearing insert 18 may be embodied with locking features such as keying tabs 26 and corresponding keying slots 28 to prevent rotation of the bearing insert 18 relative to the acetabular cup 12. As such, the acetabular cup 12 and the bearing insert 18 cooperate to provide an artificial bearing surface on which a natural or artificial head of a femur of a patient may bear.

The acetabular cup 12 may be constructed with an implant-grade biocompatible metal, although other materials may also be used. Examples of such metals include cobalt, including cobalt alloys such as a cobalt chrome alloy, titanium, including titanium alloys such as a Ti6Al4V alloy, and stainless steel. The surfaces of the acetabular cup 12 that engage the natural bone, such as its rounded outer surface 14, may have a porous metallic coating 20 disposed thereon to facilitate securing the cup 12 to the bone and to promote bone ingrowth for permanent fixation. The porous metallic coating 20 may be embodied as, for example, a coating or layer of cobalt-chromium alloy beads, such as a product sold by DePuy Synthes, a Johnson & Johnson Company located in Warsaw, Ind., under the trade mark POROCOAT®. Another illustrative example of a porous metallic coating that may be disposed on the outer surface 14 of the acetabular cup 12 is a coating or layer of porous titanium, such as a product sold by DePuy Synthesis under the trademark GRIPTION®.

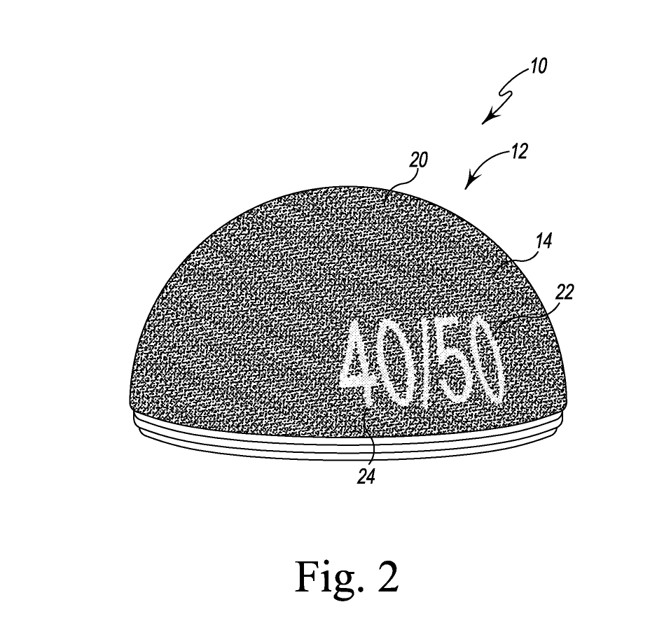

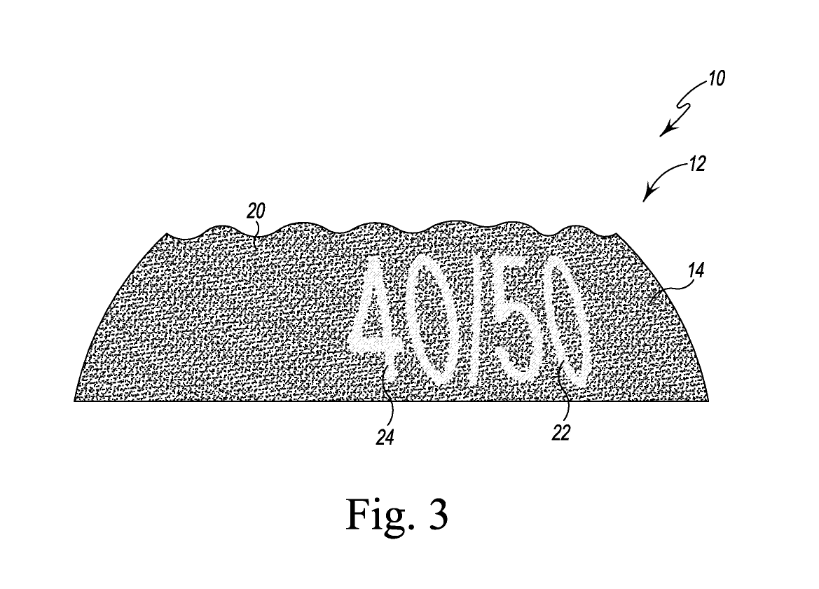

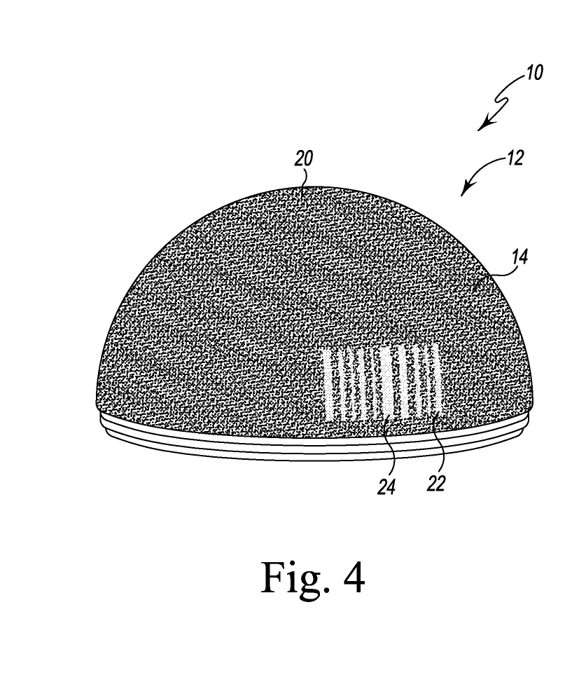

As can be seen in FIGS. 2 and 3, a marking coating 22 is disposed on the porous metallic coating 20 of the acetabular cup 12. The marking coating 22 is arranged in a pattern 24 which defines a readable indicia. In the illustrative example of FIGS. 2 and 3, the pattern 24 may be embodied as a human-readable indicia such as one or more alphanumeric characters. It should be appreciated that, as used herein, the term “alphanumeric characters” is not intended to be limited to any particular language, but rather is intended to cover letters, symbols, and numbers associated with any language. As such, the term “alphanumeric characters” not only includes the Latin letters and Arabic numbers associated with such languages as English, French, German and the like, but also the characters or symbols associated with other languages such as Japanese and Chinese. As shown in FIG. 4, the pattern 24 may take the form of any type of machine-readable indicia such as one-dimensional barcodes, two-dimensional barcodes, or any other type of machine-readable code.

The marking coating 22 is embodied as a biocompatible material having a color which creates a readable contrast to the porous metallic coating 20. One example of such a coating 22 is a white hydroxyapatite (HA) coating. In such an example, a relatively large size marking (e.g., alphanumeric characters) can be applied to the porous metallic coating 20 by spraying it onto and into the porous structure of the metallic coating 20. In an exemplary embodiment, a stencil or the like may be used to contain the outer boundaries of the marking. In another embodiment, a computer-operated spray system may be programmed to apply the marking.

In the exemplary embodiment described herein, the hydroxyapatite coating 22 may be applied to the porous metallic coating 20 via thermal spraying. One particularly useful commercially-accepted method of thermal spraying hydroxyapatite is plasma spraying. By using a thermal spraying process, such as plasma spraying, a dense white hydroxyapatite marking (e.g., alphanumeric characters) may be sprayed into porous metallic coating 20 without physical disruption to the porous metallic coating's function of bone attachment and ingrowth. Use of hydroxyapatite may be further desirable based on its biocompatibility and bone ingrowth characteristics.

In other embodiments, the marking coating 22 may be made from other resorbable materials. In such an embodiment, the resorbable marking coating 22 performs its function of being readable—i.e., human-readable or machine-readable—prior to implantation, but then is absorbed into the host (i.e., into the patient) subsequent to implantation. An example of such a resorbable material includes tricalcium phosphate. Similarly to the hydroxyapatite markings described above, such a resorbable marking made from tricalcium phosphate may be applied to the porous metallic coating 20 of the acetabular cup 12 via spraying such as thermal spraying (e.g., plasma spraying). Other examples of resorbable materials include resorbable polymers such as poly(L-lactide) and poly(lactic-co-glycolic acid).

The indicia produced by use of the marking coating 22 described herein can be used for many different functions. For example, the indicia can be an identification marking to indicate the manufacturer, lot code, implant size, or regulatory codes (e.g., CE mark). In one embodiment, some of the above information can be encoded in a barcode positioned on a non-bone engaging surface (e.g., the inner surface 16 or the rim of the acetabular cup 12) with only the implant's size being coated (e.g., sprayed) in human-readable alphanumeric characters onto the porous metallic coating 20 of the outer surface 14 of the acetabular cup 12. Such an embodiment provides an easily identifiable visual confirmation to the surgeon of the implant's size during a surgical procedure. In such a case, use of large, easily visible numbers indicative of the size of the implant provide the surgeon with a quick and easy way to match the size of the implant to the size of the surgical reamer and other instruments.

It should be appreciated that use of the marking coating 22 to create readable indicia (e.g., sprayed hydroxyapatite alphanumeric characters) on the porous metallic coating 20 of the acetabular cup 12 eliminates the need to etch such indicia into the porous metallic coating 20. In doing so, the indicia may be more readily legible to the surgeon since the colored (e.g., white) coating 22 creates a better contrast against metallic coating 20 than does etching. Moreover, etching removes a portion of the porous metallic coating 20 thereby reducing the size of the implant's overall porous coated surface relative to an implant that has been sprayed with the marking coating 22 since the marking coating 22 is applied on top of the porous metallic coating 20.

Although the concepts described herein have been illustrated in the context of the acetabular cup 12, it should be appreciated that such concepts are useful in regard to numerous other types of orthopaedic implants. For example, the porous coated bone-engaging surfaces of knee implants, such as femoral components, tibial trays, knee stems, and sleeves, may be marked using the marking coatings 22 described herein. Similarly, the porous coated bone-engaging surfaces of shoulder implants, such as humeral stem components, proximal body components, humeral cup components, glenoid components, and metaglene components, may be marked using the marking coatings 22 described herein. Moreover, the porous coated bone-engaging surfaces of other hip components beyond the acetabular cup, such as femoral hip stem components, may be marked using the marking coatings 22 described herein. The porous coated bone-engaging surfaces of the talar component and the tibial component of an ankle prosthesis may also be marked using the marking coatings 22 described herein. In short, it is contemplated to use the concepts of the present disclosure to mark the porous coated bone-engaging surfaces of numerous different types of orthopaedic implants.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure as defined by the appended claims.

1. An orthopaedic hip implant comprising:
an acetabular cup having a rounded outer surface configured to be implanted into a surgically-prepared surface of a patient's acetabulum,
a porous metallic coating disposed on the rounded outer surface of the acetabular cup, and
a hydroxyapatite coating disposed on the porous metallic coating in a pattern which defines a readable indicia.

2. The orthopaedic hip implant of claim 1, wherein the hydroxyapatite coating is disposed on the porous metallic coating in a pattern which defines a human-readable indicia.

3. The orthopaedic hip implant of claim 2, wherein the hydroxyapatite coating is disposed on the porous metallic coating in a pattern which defines a human-readable indicia indicative of the size of the acetabular cup.

4. The orthopaedic hip implant of claim 1, wherein the hydroxyapatite coating is disposed on the porous metallic coating in a pattern which defines a machine-readable indicia.

5. The orthopaedic hip implant of claim 4, wherein the machine-readable indicia comprises a barcode.

6. The orthopaedic hip implant of claim 1, wherein the hydroxyapatite coating comprises a plasma-sprayed hydroxyapatite coating.

7. An orthopaedic implant comprising:
a metallic joint prosthesis having an outer surface configured to be implanted into a surgically-prepared surface of a bone of a patient,
a porous metallic coating disposed on the outer surface of the joint prosthesis, and
a hydroxyapatite coating disposed on the porous metallic coating in a pattern which defines a readable indicia.

8. The orthopaedic implant of claim 7, wherein the hydroxyapatite coating is disposed on the porous metallic coating in a pattern which defines a human-readable indicia indicative of the size of the metallic joint prosthesis.

9. The orthopaedic implant of claim 7, wherein the hydroxyapatite coating is disposed on the porous metallic coating in a pattern which defines a machine-readable indicia.

10. The orthopaedic implant of claim 9, wherein the machine-readable indicia comprises a barcode.

11. The orthopaedic implant of claim 7, wherein the hydroxyapatite coating comprises a plasma-sprayed hydroxyapatite coating.

12. The orthopaedic implant of claim 7, wherein the metallic joint prosthesis is selected from the group consisting of a hip implant, a knee implant, a shoulder implant, and an ankle implant.

13. An orthopaedic hip implant comprising:
an acetabular cup having a rounded outer surface configured to be implanted into a surgically-prepared surface of a patient's acetabulum,
a porous metallic coating disposed on the rounded outer surface of the acetabular cup, and
a resorbable coating disposed on the porous metallic coating in a pattern which defines a readable indicia.

14. The orthopaedic hip implant of claim 13, wherein the resorbable coating is disposed on the porous metallic coating in a pattern which defines a human-readable indicia.

15. The orthopaedic hip implant of claim 14, wherein the resorbable coating is disposed on the porous metallic coating in a pattern which defines a human-readable indicia indicative of the size of the acetabular cup.

16. The orthopaedic hip implant of claim 13, wherein the resorbable coating is disposed on the porous metallic coating in a pattern which defines a machine-readable indicia.

17. The orthopaedic hip implant of claim 16, wherein the machine-readable indicia comprises a barcode.
